(19) United States
(12) Patent Application Publication
(10) Pub. No.: US 2026/0363757 A1
(43) Pub. Date: Nov. 19, 2026

(54) ELECTROSURGICAL DEVICE AND METHOD

(71) Applicant: M. I. ADVANCED THERMOSURGERY, INC., Hanover, NH (US)

(72) Inventors: KORNEL HORVATH, Budaors (HU); GERGO CSIKOS, Budapest (HU)

(21) Appl. No.: 19/126,019

(22) PCT Filed: Nov. 1, 2023
(86) PCT No.: PCT/US2023/078389

§ 371 Date: Apr. 30, 2025

Related U.S. Application Data
(60) Provisional application No. 63/381,869, filed on Nov. 1, 2022.

(51) Int. Cl. A61B 18/14; A61B 18/00

(52) U.S. Cl. CPC A61B 18/1447; A61B 2018/00601; A61B 2018/00619; A61B 2018/1455

(57) ABSTRACT

The present disclosure includes a number of electrosurgical devices and methods for sealing and transecting tissue, such as blood vessels and intestines, wherein the sealing of tissue is provided by tissue fusion performed by delivering energy to the tissue. The present disclosure also includes devices and methods for welding tissue. An exemplary device may include an elongate shaft carrying a first fixed clamping component and a second moveable clamping component. These components may each respectively have a first tissue-engaging surface and a second tissue engaging surface and a bi-polar electrode arrangement in the tissue-engaging surfaces. Additionally, the devices described may include an energy supply that comprises a controller and at least one RF source for delivering current and where the bi-polar electrode arrangement is operatively connected to the controller.

### PRIORITY CLAIM

This patent application claims priority to U.S. provisional patent application No. 63/381,869, titled “ELECTROSURGICAL DEVICE AND METHOD,” and filed on Nov. 1, 2022, which is herein incorporated by reference in its entirety.

### FIELD OF THE INVENTION

The present invention relates to electrosurgical devices and methods for sealing and transecting tissue, such as blood vessels and intestines, wherein the sealing of tissue is provided by tissue fusion performed by delivering energy to the tissue.

### SUMMARY OF THE INVENTION

The present disclosure includes devices and methods for welding tissue. In one example of such a device, the device includes an elongate shaft carrying a first fixed clamping component and a second moveable clamping component, each respectively having a first tissue-engaging surface and a second tissue-engaging surface and a bi-polar electrode arrangement in the tissue-engaging surfaces.

The devices described herein can include an energy supply that comprises a controller and at least one RF source for delivering current and where the bi-polar electrode arrangement is operatively connected to the controller.

The controller can be adapted to sense at least one electrical parameter of current delivery consisting of impedance, capacitance, and/or phase angle to sense the thickness of engaged tissue when the first and second clamping components engage tissue. In another variation, the controller can be adapted to multiplex current delivery among various elements of the opposing polarity electrodes. The controller can be adapted to modulate current delivery to the bi-polar electrode arrangement in response to signals from at least one temperature sensor. Alternatively, controller can be adapted to sense at least one electrical parameter of current delivery consisting of impedance, capacitance, and/or phase angle to sense an effective tissue weld. The controller can also terminate current delivery when a sensed electrical parameter indicates said effective tissue weld.

In a variation of the device, the device includes a motor drive configured to move the second clamping component relative to the fixed first clamping component. The controller can be adapted to actuate the motor drive to move the clamping component at a variable rate. In additional variations, the controller is adapted to actuate the motor drive to move the second clamping component at a first closing rate until the clamping components are spaced apart by a selected distance, followed by a second closing rate to compress a tissue between the clamping components to a thickness of less than 0.5 mm (or any other range/distance as required). The controller can also stop the movement of the second clamping component when the thickness of the engaged tissue is within a preselected range. Additionally, or in the alternative, the controller stops the movement together of the second clamping component controller when the controller senses at least one electrical parameter that indicates the thickness of the tissue is within a preselected range. The controller can also actuate the motor drive to move apart the clamping components after the controller senses at least one electrical parameter indicating an effective tissue weld.

### BRIEF DESCRIPTION OF THE DRAWINGS

FIG. 1 is a cut-away perspective view of a surgical instrument corresponding to the invention, illustrating a general overall view of the instrument with a handle and an elongated shaft and working end with a bi-polar electrode arrangement.

FIG. 2 is a perspective view of the working end of the instrument of FIG. 1, showing a first fixed clamp member and an axially-slidable second clamp member with an outer sleeve of the shaft in phantom view.

FIG. 3 is a perspective view of an axially moveable blade element separated from the shaft and first and second clamp members of FIGS. 1 and 2.

FIG. 4 is a perspective view of the working end and first fixed clamp member of FIG. 2 with the axially-slidable second clamp member removed to show a variation of a bi-polar electrode arrangement.

FIG. 5 is a perspective view of the working end and the axially-slidable second clamp member with the removed to show a variation of a bi-polar electrode arrangement, with the first fixed clamp member removed to show a variation of a bi-polar electrode arrangement.

FIG. 6A is a schematic side view of the working end of FIG. 2 with the axially-slidable second clamp member in a retracted position for capturing tissue, with the outer sleeve of the shaft in transparent view.

FIG. 6B is a side view of the working end of FIG. 6A with the axially-slidable second clamp member in an extended position for engaging and compressing tissue.

### DETAILED DESCRIPTION OF THE INVENTION

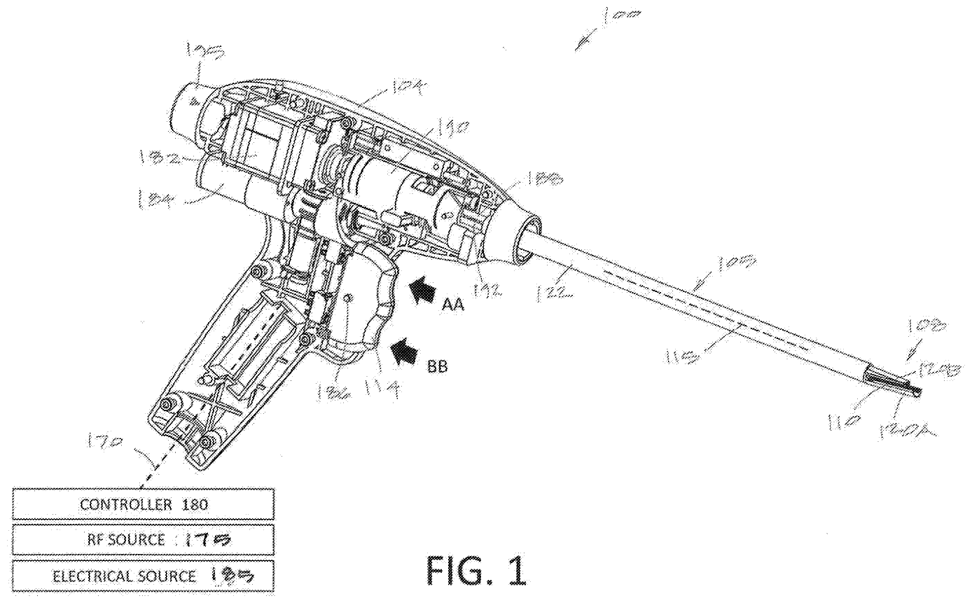

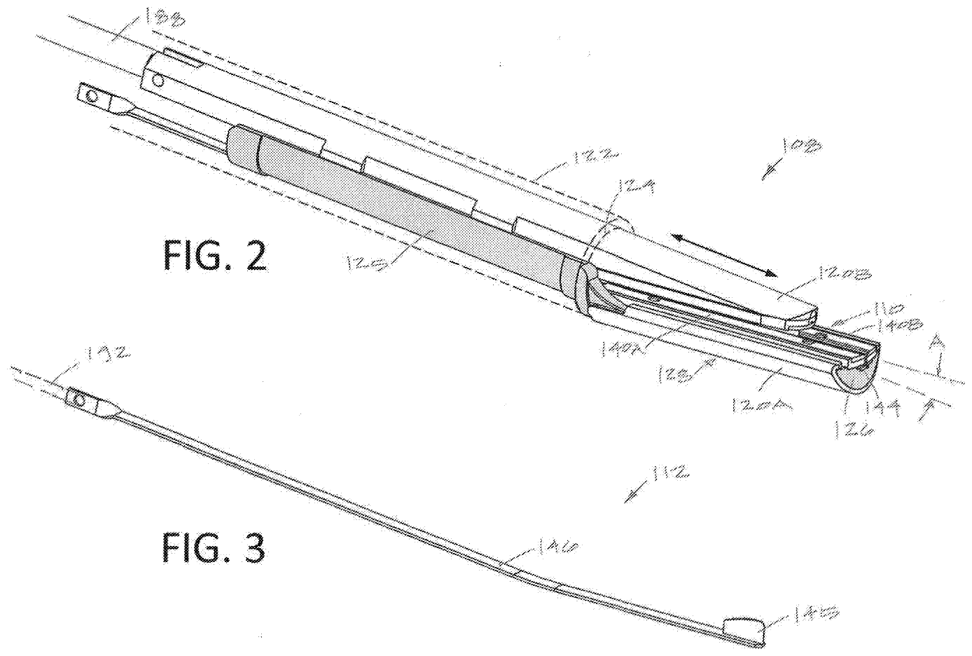

FIG. 1 illustrates an electrosurgical device 100 corresponding to the invention, which is adapted for sealing and transecting tissue such as a hollow tubular body organ (e.g., blood vessels, intestines, and the like). In general, the electrosurgical device 100 has a handle 104 coupled to an elongated shaft assembly 105 with a distal working end 108 comprising a tissue-clamping assembly and a bi-polar electrode arrangement 110 for engaging tissue and applying energy to the tissue. The device 100 further includes an axially movable blade assembly 112 (FIG. 2) for transecting the tissue after or before sealing the tissue with electrode arrangement.

As can be seen in FIG. 1, the electrosurgical device 100 has a handle 104 with a pistol grip and an actuator or trigger 114 for actuating the device, as further described below. The elongated shaft assembly 105 extends about a longitudinal axis 115 to the working end 108 that includes first and second tissue-clamp members 120A, 120B that are adapted to capture and clamp tissue under high compression. The tissue-clamp members 120A and 120B are further configured to apply energy from the bi-polar electrode arrangement 110 for thermally welding the engaged tissue. The shaft 105 of the device 100 can have any suitable length and typically has a straight, rigid configuration. However, a curved shaft configuration or an articulated shaft configuration falls within the scope of the invention.

Referring to FIGS. 1 and 2, shaft assembly 105 comprises an outer sleeve 122 that has an inner bore or passageway 124 therein. As shown in FIG. 2, the first tissue clamp member 120A has a proximal portion 125 that is fixed in the bore 124 of the outer sleeve 122 of the shaft assembly.

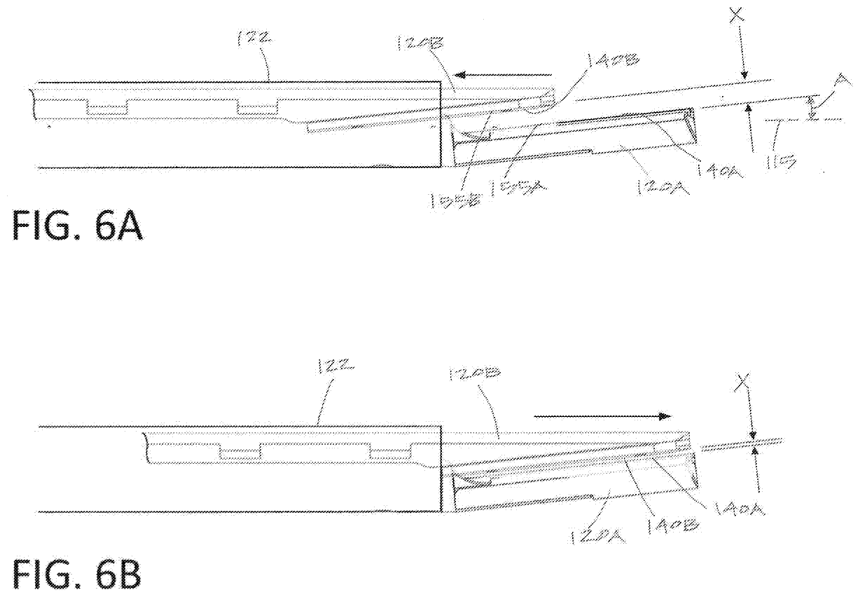

The shaft assembly 105 and the first tissue clamp member 120A are fixed together by a press fit, adhesives, welding, or the like. Typically, the proximal portion 125 of the first clamp member 120A is a molded plastic that extends through the core 126 of the distal portion 128 of the clamp member 120A. As can be seen in FIGS. 2 and 6A-6B, the tissue-engaging surface 140A of the clamp member 120A is angled at angle A relative to the longitudinal axis 115 of the shaft assembly 105 from about 5° degrees to 20°.

FIG. 2 also shows the second moveable clamp member, 120B, is axially translatable in the bore 124 of the outer sleeve 122 of the shaft assembly 105 between a retracted position and an extended position, with FIG. 2 showing the second clamp member 120B in an intermediate position. FIGS. 6A and 6B show the second clamp member 120B in a partially retracted position (FIG. 6A) and a fully extended position (FIG. 6B). The second clamp member 120B has a surface 140B that opposes the surface 140A of the first clamp member 120A. The second tissue engaging surface 140B is also angled at angle A relative to a longitudinal axis 115 of shaft assembly 105 to match the angle of the surface 140A of clamp member 120A. As will be described further below, both the tissue-engaging surfaces 140A, 140B of the clamp members 120A, 120B carry the bi-polar electrode arrangement 110. Referring to FIG. 2, it can be understood that when the second clamp member 120B is moved toward its distal or extended position, the first and second tissue-engaging surfaces 140A, 140B, and 140B move closer together to capture and clamp tissue under very high compression forces.

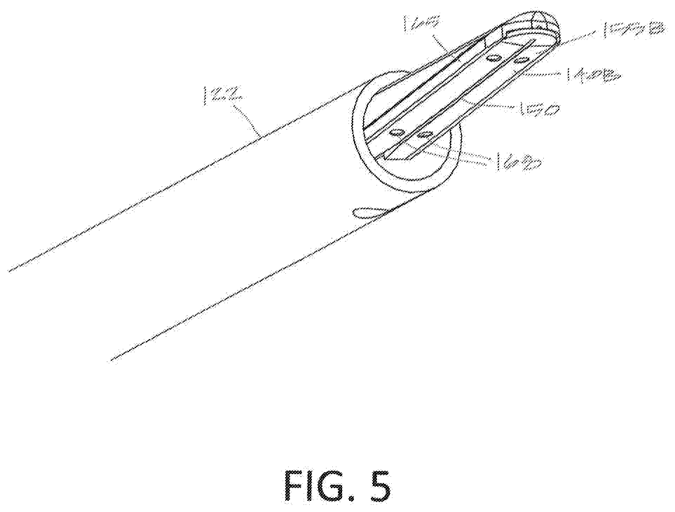

In FIGS. 2, 3, and 4, it can be seen that first clamp member 120A has a central T-shaped channel 144 in which the blade assembly 112 and cutting blade 145 are configured to move from a retracted position to an extended position and back to the retracted position to transect tissue engaged between the clamp members 120A and 120B. The blade assembly 112, as shown in FIG. 3, comprises an elongate flexible, flat metal member 146 and the distal cutting blade 145 welded thereto. The central T-shaped channel 144 in the first clamp member 120A is thus configured to receive the slidable flat metal member 146 and cutting blade 145 of the blade assembly 112. FIG. 3 shows the blade assembly 112 in a flexed position as when the blade assembly is advanced through a T-shaped receiving channel 144 in the angled, first clamp member 120A as in FIG. 2. As can be understood from FIG. 2, the elongate flexible member 146 of the blade assembly needs to flex as it moves from a retracted position in the shaft assembly 105 toward a fully extended position within the angled first clamp member 120A. FIG. 5 shows that the movable, second clamp member 120B also includes a central slot 150 for receiving the cutting blade 145 of the blade assembly 112 when the first and second clamp members 120A, 120B have engaged and clamped targeted tissue.

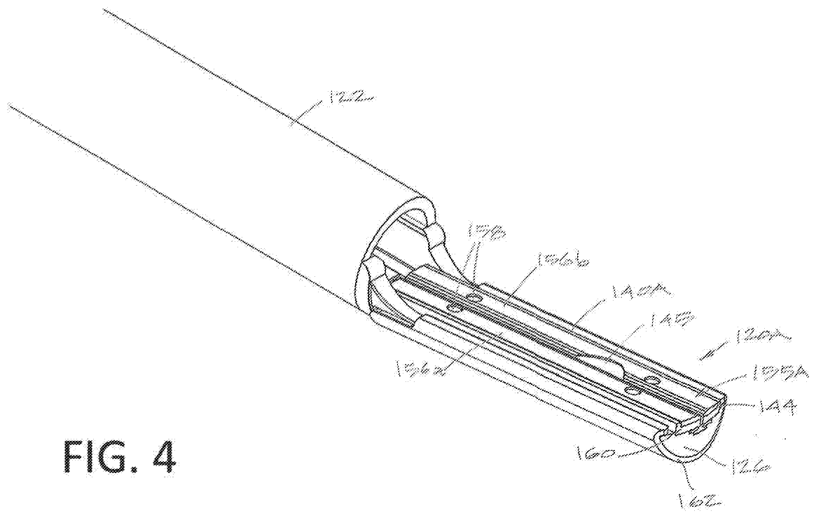

FIG. 4 shows the outer sleeve 122 and first clamp member 120A with the second clamp member 120B removed. In the view of FIG. 4, it can be seen that the first clamp member 120A has a first polarity electrode 155A comprising electrode elements 156a and 156b on either side of channel 144, which receives the cutting blade 145. The electrode elements 156a and 156b are secured by pins 158 in an insulator base 160, which is typically a ceramic. In a variation, the ceramic is fixed in place on the polymer core 126 by adhesives or is locked in place with a metal outer sheath 162, as shown in FIGS. 2 and 4. The metal sheath 162 is adapted to strengthen the polymer core 126 of the first clamp member 120A against flexing when tissue is clamped under very high pressure by distal extension of the second clamp member 120B to clamp and compress tissue.

FIG. 5 shows the outer sleeve 122 and the movable second clamp member 120B with the first clamp member 120A removed. In FIG. 5, it can be seen that the second clamp number 120B has a U-shaped second polarity electrode 155B that extends around the central slot 150 that receives the cutting blade 145 when the second clamp member 120B is moved toward an extended position. The U-shaped electrode 155B is secured to insulator member 165 by pins 168, and the insulator member 160 is fixed to the second clamp member 120B by adhesives or other suitable means. Thus, the opposing surfaces 140A and 140B of the first and second clamp members 120A, 120B comprise the bipolar electrode arrangement 110, which is connected by cable 170 to an RF source 175 and controller 180, as indicated in FIG. 1.

Now referring to FIG. 1, device 100 carries first and second electric motors 182 and 184, as will be described below, that are connected through cable 170 to an electrical source 185 and controller 180. In a variation, the controller 180 is adapted to control (i) movement of the second clamp member 120B from a retracted position to an extended position to clamp tissue, (ii) delivery of RF energy to the electrode agreement 110, (iii) movement of the blade assembly 112 forward and back to transect tissue, and (iv) movement of the second clamp member 120B from the extended position to a retracted position to release engagement of the welded and transected tissue.

In FIG. 1, the handle 104 is configured with an actuator 114 adapted to rotate around pivot pin 186 to actuate the mechanisms and functions as described above. In a variation, the single actuator 114, in cooperation with the controller 180, can operate all the needed functions. First, the actuator 114 can be actuated with pressure in the direction of arrow AA of FIG. 1, which then actuates motor drive 182 to move the second clamp member 120A from a retracted position toward an extended position to clamp tissue between the tissue-engaging surfaces 140A, 140B and electrodes 155A and 155B. In this variation, the motor drive 182 and a rotation-to-linear mechanism 190 then axially drive the drive member 188 in the bore 124 of the sleeve 122, which is connected to the second clamp member 120A as can be seen in FIGS. 1 and 2. The extension of the drive member 188 and the second clamp member 120A by the motor drive 182 can be stopped by a sensor mechanism that senses pressure on the drive member 188, that in turn indicates that the clamped tissue has been compressed to a selected, minimal thickness. A type of sensor mechanism can comprise an algorithm in the controller 180 that monitors voltage increases required to advance the second clamp member toward an extended position. The controller 180 can then determine that the targeted tissue compression level has been achieved, and the controller 180 thereafter can stop the motor drive 182 and lock the second clamp member 120A in its then extended position. The controller 180 can provide an audio or visual signal to the physician that the tissue is properly engaged and compressed. Thereafter, a second movement of the actuator 114 in direction AA will result in the electrodes 155A and 155B being energized to thereby deliver energy and seal or weld the engaged tissue. Determination of optimal tissue sealing can be determined by the controller 180 monitoring impedance and/or temperature from temperature sensors (not shown) as known in the art. After impedance and/or temperature reaches a selected level, the controller 180 can terminate energy delivery from the RF source 175 and provide an audio or visual signal. After the RF energy delivery had been terminated and the tissue has been welded, the physician can actuate the actuator 114 again in direction AA for a third time to actuate the second motor drive 184 to move the cutting blade 145 from its retracted position to an extended position and back to the retracted position to transect the tissue. A drive rod 192 is shown in FIGS. 1 and 2, which couples the motor drive 184 and a linear actuator to the blade assembly 112. The controller 180 again monitors the actuation of the cutting blade and provides an audio or visual signal that the tissue has been transected. Thereafter, the physician can actuate the actuator 114 in the direction indicated by arrow BB in FIG. 1, which will retract the second clamp member 120B from its extended position to a retracted position to thereby disengage from the welded and transected tissue. In this variation, the single trigger 114 is adapted for actuating the various functions of the device, but it should be appreciated that multiple different actuators can be used for each function.

FIGS. 6A and 6B show the working end 108 of the device 100 with the moveable second clamp member in a partially retracted position (FIG. 6A) and a fully extended position (FIG. 6B) when clamping tissue. In a variation, when engaging tissue, the distal movement of the second member 120A can be stopped when a selected dimension X is achieved between the surfaces 140A and 140B of the first and second members 120A and 120B, for example, and dimension between 0.1 mm and 1.0 mm which would be selected by the physician after observing the targeted tissue. The controller 180 then can be set to move the second clamp m member 120B to clamp tissue to the selected dimension X. The stroke of the axially-moveable second member 120A between a fully retracted position and an extended position is between 1 cm and 10 cm. The dimension X in FIG. 6A between and perpendicular to the first and second tissue-engaging surfaces 140A, 140B in the retracted position is at least 2 mm. The dimension X between the first and second tissue-engaging surfaces 140A, 140B in the extended position of FIG. 6A is less than 1.0 mm.

In a variation, controller 180 can include a mechanism wherein the movement of the blade assembly 112 can only occur when the second movable clamp member 120B has been extended to a suitable position for clamping and compressing tissue. In another variation, a single actuation of trigger 114 can cause the controller 180 to sequentially actuate all the steps listed above to clamp, weld, and transect the tissue.

In another variation, a rotatable adjusting knob or grip 195 can be provided at the proximal surface of handle 104 that allows the physician to adjustment of the targeted dimension X between the first and second tissue-engaging surfaces 140A and 140B as shown in FIG. 6B.

In another variation of a tissue-clamping assembly, energy delivery for tissue welding can be provided by resistive heating elements, inductive heating elements, ultrasound transducers, light energy emitters, and the like. Further, a circular stapling mechanism can be provided in the clamping assemblies as is known in the art in combination with the thermal welding mechanism described above.

Although particular embodiments of the present invention have been described above in detail, it will be understood that this description is merely for purposes of illustration and the above description of the invention is not exhaustive. Specific features of the invention are shown in some drawings and not in others, and this is for convenience only, and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

All references, including publications, patent applications, and patents, cited herein are hereby incorporated by reference to the same extent as if each reference were individually and specifically indicated to be incorporated by reference and were set forth in its entirety herein.

1. An electrosurgical instrument, comprising:
a handle coupled to an elongated shaft having a longitudinal axis;
the elongated shaft configured with a first member with a first tissue-engaging surface having a first fixed angle relative to the longitudinal axis and a second member with a second tissue-engaging surface having a second fixed angle relative to the longitudinal axis, the first member located at a distal end of the elongated shaft and the second member being axially-moveable;
a mechanism for moving the second member between a retracted position and an extended position wherein the retracted position has an increased space between the first tissue-engaging surface and the second tissue-engaging surface for capturing tissue and wherein the extended position has a decreased space between the first tissue-engaging surface and the second tissue-engaging surface for clamping tissue; and
a first bi-polar electrode and a second bi-polar electrode disposed in spaced apart relation in the first tissue-engaging surface and the second tissue-engaging surface.

2. The electrosurgical instrument of claim 1, wherein the first fixed angle is between 5° and 20°.

3. The electrosurgical instrument of claim 1, wherein the second fixed angle is between 5° and 20°.

4. The electrosurgical instrument of claim 1, wherein a stroke of the second member when axially moved between the retracted position and the extended position is between 1 cm and 10 cm.

5. The electrosurgical instrument of claim 1, wherein a dimension between and perpendicular to the first tissue-engaging surface and the second tissue-engaging surface in the retracted position is at least 2 mm.

6. The electrosurgical instrument of claim 1, wherein a dimension between and perpendicular to the first tissue-engaging surface and the second tissue-engaging surface in the extended position is less than 1.0 mm.

7. The electrosurgical instrument of claim 1, wherein the first tissue-engaging surface and second tissue-engaging surface carry opposing polarity electrodes.

8. The electrosurgical instrument of claim 1, wherein the second member is axially moveable between the retracted position and extended position by a motor drive.

9. The electrosurgical instrument of claim 1, further comprising an axially-moveable blade assembly adapted to move axially between a retracted position and extended position in the first member and the second member.

10. The electrosurgical instrument of claim 9, wherein the axially-moveable blade assembly is moved between the retracted position and extended position by a motor drive.

11. The electrosurgical instrument of claim 9, wherein the axially-moveable blade assembly includes a cutting blade element that is slidable in a channel in the first member.

12. The electrosurgical instrument of claim 9, wherein the axially-moveable blade assembly includes an elongate flexible element extending through the elongated shaft.
